(19) United States
(12) Patent Application Publication
(10) Pub. No.: US 2020/0040373 A1
(43) Pub. Date: Feb. 6, 2020

(54) Method of Screening Skin Products

(71) Applicant: The Procter & Gamble Company, Cincinnati, OH (US)

(72) Inventors: Karl Shiqing WEI, Mason, OH (US); Ji-Quan NMN LIU, Singapore (SG)

(21) Appl. No.: 16/055,604

(22) Filed: Aug. 6, 2018

(51) Int. Cl. C12Q 1/02; A61B 5/00

(52) U.S. Cl. CPC C12Q 1/025; C12Q 1/04; A61B 5/441

(57) ABSTRACT

A method evaluating a skin cleansing composition for an ability to treat a skin condition, can include a) identifying a target skin condition or lack of target skin condition on a skin sample; b) taking a baseline measurement of a Microbial index of Skin Health via a Microbial index of Skin Health Method; c) performing a wash protocol with the cleansing composition via the Wash Protocol Method; and d) taking a second measurement of the Microbial Skin Health Measurement after the Wash Protocol; wherein an increase from the baseline Microbial Skin Health Index of 5 or more signifies the cleansing product is efficacious for treatment of the identified skin condition.

### FIELD OF THE INVENTION

This application is directed to methods of evaluating skin products for the ability to treat a skin condition.

### BACKGROUND OF THE INVENTION

Skin is a complex, multi-layered and dynamic system that provides a protective covering defining the interactive boundary between an organism and the environment. It is the largest organ of the body and is vitally important to both our health and our self-image. The skin comprises three principal layers, the epidermis, the dermis, and a layer of subcutaneous fat.

Adding to skin's complexity is the desire to keep the skin healthy. Some skin conditions can impact the skin's microbiota and vice versa. Unfortunately, skin microbiota is diverse and it can be difficult to understand how particular skin conditions impact the microbiota and how to utilize such information to screen for cleansers which can help treat the skin condition. As such, there is a need for improved methods to screen skin products.

### SUMMARY OF THE INVENTION

A method evaluating a skin cleansing composition for an ability to treat a skin condition, can include a) identifying a target skin condition or lack of target skin condition on a skin sample; b) taking a baseline measurement of a Microbial index of Skin Health via a Microbial index of Skin Health Method; c) performing a wash protocol with the cleansing composition via the Wash Protocol Method; and d) taking a second measurement of the Microbial Skin Health Measurement after the Wash Protocol; wherein an increase from the baseline Microbial Skin Health Index of 5 or more signifies the cleansing product is efficacious for treatment of the identified skin condition.

### BRIEF DESCRIPTION OF THE DRAWINGS

FIG. 1 is graphical depiction of several bacteria and their relative abundance; and

FIG. 2 is a graph showing the top 25 most discriminatory bacteria.

### DETAILED DESCRIPTION OF THE INVENTION

Skin is a complex, multi-layered and dynamic system that provides a protective covering defining the interactive boundary between an organism and the environment. It is the largest organ of the body and is important to our health. The skin hosts a variety of bacteria, known as the microbiota. Examples of some bacteria that can be found on skin include: Staphylococcus, Micrococcus, Corynebacterium, Propionibacterium, and Neisseria.

Symbiotic bacteria on the human body have significance for the health of the human body. The disruption of these bacteria can be a symptom of an underlying condition. Thus, the make-up of the types of bacteria and the respective density of each bacteria can speak to the current state of the skin. The skin microbiota tends to be fairly stable. Thus, when shifts occur due to a skin condition, these shifts can potentially be used to determine which condition is present and whether a product being administered to the afflicted person and/or skin improves the skin condition. Thus, it is believed that by looking at the microbiota of healthy skin and compromised skin, and determining the differences between the two, a subset of the microbiota can be identified which correlates to the skin condition. If one can correlate a particular bacteria set to a particular skin condition, then that bacteria set could be used to determine if a product can be used to improve skin having the underlying condition.

So, once a skin condition of interest is identified, healthy and inflicted skin samples can be collected. This can be done, for example, by relying on people to self-diagnosis the skin condition or diagnosis from a professional or physician that their skin has the target condition or is healthy. For example, an individual may notice dry and/or flaky patches of skin or, even, redness, which could all be used to self-diagnose a skin condition. The parameters for each population of subjects and how they are selected can be optimized to the desires of those running the test. Once the requisite number of skin samples is identified for each set, the skin samples can be analyzed for any bacteria, bacterial genera, or combination thereof which correlates to the target skin condition. This can be done, for example, by a three-part process: collection, sequencing, and analyzing.

Collection of a skin sample can be done by any known method. For example, skin samples can be taken with a cotton swab by swabbing on a desired area of skin about 30 times. The desired area of skin could change depending on the target skin condition. For example, for skin conditions with lesions, the skin collection may take place from the lesion, near the lesion, or both. For skin with both lesion and non-lesion areas, collecting information from the non-lesion area can provide helpful information on the breadth of the underlying skin conditions. The swabbing can be in any desired fashion, like a circular motion or a back and forth motion. The cotton swab can first be treated with a buffer to help in the collection of skin cells. One example of how skin cells can be collected is found in the Collection Method described below. Additional methods of skin cell collection can include, for example, D-Squame, tape stripping, skin biopsy, or a combination thereof, in line with standard collection procedures.

Once the skin samples are collected, they can be tested. The testing of the skin samples can include sequencing of the DNA on the skin samples. This can be done, for example, by 16S ribosomal RNA sequencing per the known procedure. An example of a sequencing method can be found below. The sequencing can include the relevant regions, like V1-V4, for example, V1-V3. This type of sequencing can be utilized to characterize a bacterial community.

Once the sequencing is complete, the data can be analyzed, for example, using a random forest model. A program, like Parallel-META 3, can be used to analyze the data utilizing the model and output the bacteria and/or genera of the samples. The output can be mined to look for shifts or changes in the bacteria types and/or abundances between the healthy samples and the samples with the skin condition which can lead to the discovery of target microbiota for the target skin condition.

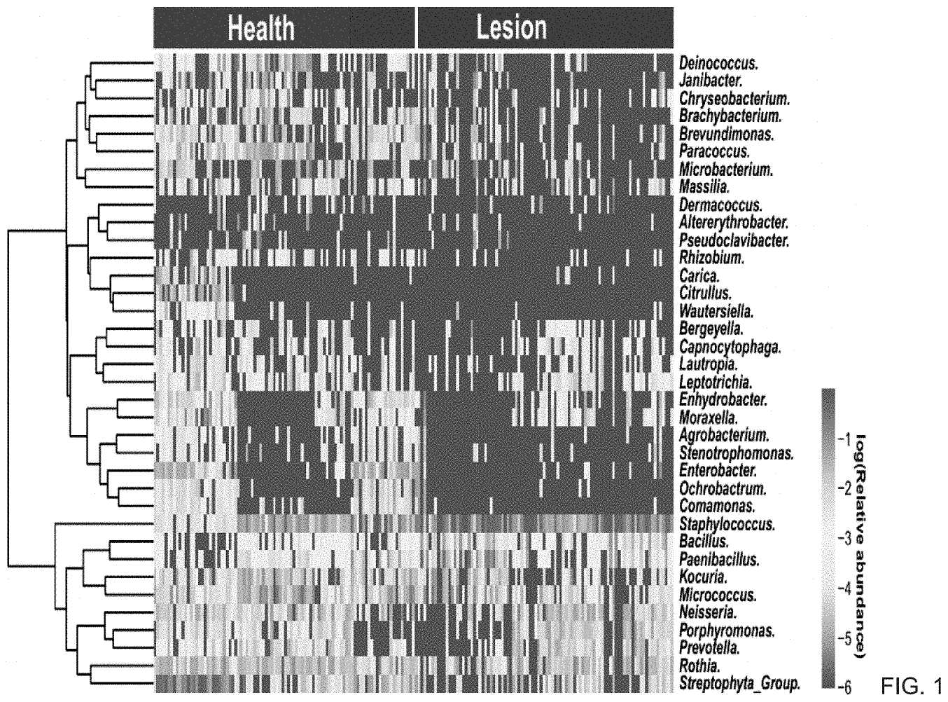

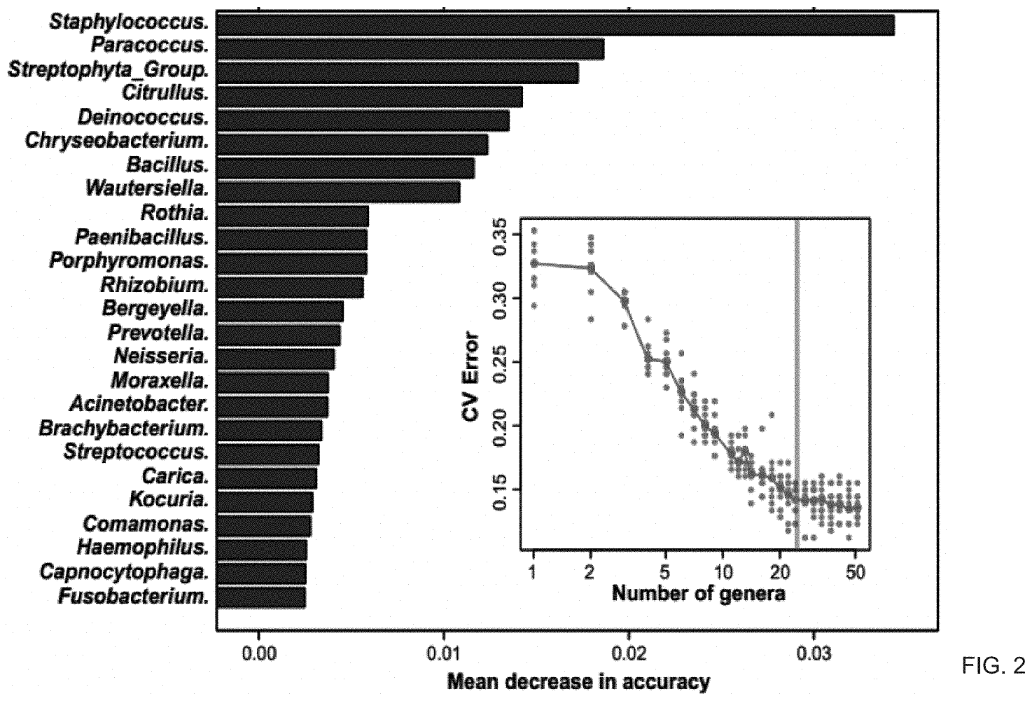

For example, skin samples of healthy skin and skin with atopic dermatitis were taken from individuals in two cities in China and one in the United States. The microbiota of these individuals was studied. The microbiota from both the healthy and atopic dermatitis individuals was utilized to build a Random Forest model, using profiles of the taxa at six different phylogenetic levels (from genus to phylum). The area under the curve (AUC) was maximized at the genus level and performance of the model was minimal after the top 25 most discriminatory bacterial genera were included. These bacteria include: Fusobacterium, Capnocytophaga, Haemophilus, Comamonas, Kocuria, Carica, Streptococcus, Brachybacterium, Acinetobacter, Moraxella, Neisseria, Prevotella, Bergeyella, Rhizobium, Porphyromonas, Paenibacillus, Rothia, Wautersiella, Bacillus, Chryseobacterium, Deinococcus, Citrullus, Streptophyta Group, Paracoccus and Staphylococcus (see FIGS. 1 and 2). This bacterial cluster makes-up the target for the microbial index of skin health (MiSH), which was obtained through “probability of Random Forest modeling” based on the similarity algorithm after model distinguishing, and multiplying the value by 100. The skin microbial health scale refers to 0-100 value of MiSH, where the higher the number the more likely it is healthy. The value of MiSH 100 refers to the probability that a given skin sample is fully healthy.

To test its spatial scalability, performance of MiSH was compared among: (i) matching sites of individuals in a city, (ii) among individuals in a city, (iii) among individuals from the two Chinese cities, and finally (iv) among individuals from all the three cities. A step-wise reduction of diagnosis accuracy from 0.98, 0.97, 0.93 to 0.90 suggests that introduction of microbiota heterogeneity at each additional spatial scale would reduce model performance. Consistent with this, for healthy samples, the size of the core microbiome (defined as the number of genera found in >50% of samples) follows a similar downward trend; this indicates an effect based on number of accumulated samples rather than geography, because the reduction of the core microbiome size is correlated with the number of samples rather than the number of cities. For lesion samples, the core microbiome size largely plateaued when extending the scale beyond a single city, and is smaller than the core microbiome of the healthy samples at all scales, suggesting a conserved set of atopic dermatitis markers that is largely independent of the number of cities included. This observation explains why this diagnosis model scales over large geographic distances.

Once a bacteria cluster has been identified as correlating to a particular skin condition, tests can be set up to evaluate whether a particular product impacts the cluster and thus can potentially influence the state of the skin. For example, skin samples can be collected and sequenced (as discussed in more detail above). Primers and/or probes can be identified and used for the target genera. These primers and probes could be general or specific. Afflicted skin can be treated with the target product for a specified protocol and then tested for a change in the correlated bacteria cluster. For example, a corticosteroid was applied to lesion sites every day for four weeks. MiSH of both lesion and non-lesion samples, at 20.2±17.9 and 25.1±10.4 respectively before treatment, significantly improved after the treatment (41.3±16.7, p=9.07e-6 and 40.9±19.9, p=2.07e-5 respectively; the response at lesion and non-lesion sites was statistically indistinguishable p=0.43). Moreover, the treatment moved the skin microbiota of both lesion and non-lesion sites across the body to a structure more similar to healthy samples than to lesion or non-lesion ones. These results support the use of MiSH for objectively assessing the efficacy of a product for treatment of a target condition.

In addition, three cleansing compositions were evaluated to determine if MiSH could be utilized as a screener for products for application to skin with atopic dermatitis. Skin samples were collected at baseline and again after four weeks of treatment with the cleansing composition in combination with a corticosteroid. The skin samples were analyzed and the MiSH was computed. One cleansing composition, which included zinc pyrithione, induced a microbiota change pattern that was the most similar to healthy skin. It had the largest increase in MiSH score of the 3 cleansing compositions tested. Such superior efficacy of the zinc pyrithione cleansing composition is likely due to its antibacterial activity (neither of the other two cleansers contained an antimicrobial), which kills more atopic dermatitis-associated bacteria such as S. aureus and thus changes skin microbiota to a healthier state. The change in the skin microbiome seems sufficiently sensitive to characterize and distinguish effects of different ingredients in skin cleansers, and such microbiome-based signatures can form a basis for objective comparison of treatment efficacy. In addition, it is believed the MiSH index could also be used to screen other skin products, like skin moisturizers, skin cream, make-up removers, etc.

### Collection Method

The sample collector should wash hands and wear rubber gloves to help prevent contamination. To collect skin cells for measurement, determine the desired area for testing. If a particular skin condition results in visible symptoms, like redness or lesions, samples should be taken from those visible symptomatic areas. If there are no visible symptoms, samples should be taken where such symptoms are likely to appear for the target skin condition. Mark the area from which collection will take place. For most skin conditions an area of about 8 cm² will suffice. For areas with a low biomass, a larger area, like ≥10 cm² may be needed.

Mark all of the collection tubes with the relevant information. Prepare single-use sterile flocked swabs (PurFlock Ultra) for skin sampling. Make sure the swabs do not come into contact with surfaces other than the target skin area and it corresponding tube during the collection process. Swabs have a handle and a cotton tip, but any acceptable swab or sampler could be used. To help facilitate collection of the skin sample, the tip of the swab may be treated with a buffer. For example, the tip of the swab may be immersed in a sterile solution of deionized water containing 0.15 M NaCl and 0.1% Tween 20. Excess solution can be removed by pressing the swab against the side of the tube which will be used for the collected sample. The swabs are continually rubbed across a target area horizontally and vertically for a total of about 30 seconds. The head of the swab is then cut off and placed in the appropriately marked tube. A cap is then placed on the tube to tightly seal it. The tube with the sample is then placed into an ice box or refrigerator until it can be placed in cold storage at −80° C. or can be placed directly into cold storage.

Once the skin sample has been collected, the DNA is extracted. To extract the DNA from a sample, the sample is thawed. 350 μL of phosphate buffered saline (PBS) is added to the tube containing the sample for extraction. 350 μL of AL buffer solution (from QIAGEN), 40 μL of lysozyme (10 mg/mL), 6 μL of mutanolysin (25000 U), and 300 mg of glass beads are added to the tube. The contents of the tube are mixed by vortexing. The tube is then incubated at 37° C. for one hour. The tube is then transferred to a tissue grinder (supplied by QIAGEN) and processed for 3 minutes at 26 Hz. 20 μL of protease K (from QIAGEN reagent kit) is added to the tube, then the tube is capped and shaken until homogeneous. The tube is then incubated at 56° C. for 3 hours.

The supernatant from the tube is then transferred to a new, clean tube and the swab is discarded. The beads are washed twice with 200 μL of distilled water. A ½ volume of alcohol is added to the tube and the contents are mixed until they become homogenous. Load the tube contents into a DNeasy centrifugation chromatographic column (purchased from QIAGEN) which has been placed in a clean centrifuge tube and allow it to be absorbed. Centrifuge the column at 8000 rpm/min for one minute. Discard the waste liquid which comes through the column and the centrifuge tube.

Place the chromatographic column into a clean centrifuge tube. Add 500 μL of AW1 buffer (QIAGEN), allow it to absorb into the column, and then centrifuge the column at 8000 rpm/min for one minutes. Discard the waste liquid and the collection tube. Once again, place the chromatographic column into a clean centrifuge tube. Add 500 μL of AW2 buffer, allow it to absorb into the column, and then centrifuge the column at 14000 rpm/min for 3 minutes. Discard the waste liquid and the collection tube. Allow the chromatographic column to dry at room temperature. A new column is used with each sample.

Place the dried column into a clean centrifuge tube and let stand at 37° C. for 2 minutes. Add 50 μL of AE buffer solution (QIAGEN) onto the dried column and incubate it at 37° C. for 3 minutes. Centrifuge the sample at 14000 rpm/min for 3 minutes. Repeat the above by adding 50 μL of AE buffer solution onto the dried column, incubate it at 37° C. for 3 minutes, and centrifuge the sample at 14000 rpm/min for 3 minutes. The combined elution sample contains the skin DNA ready for sequencing.

### Sequencing Method

The microbiota of the extracted DNA from the skin samples can be determined by putting it through the 16S rRNA sequencing method as known. The sequencing can be done on a target region and with a selected primer. For example, for the MiSH bacteria cluster, the regions targeted are V1-V3 and the primer is 27F/534R. In addition, the sequencing can be done by utilizing a reagent kit (Illumina Miseq 250/300). A 20 μL reaction mixture is made by combining 10 μL of Sybr green, 0.5 μL of upstream primer, 0.5 μL of downstream primer, 5 μL of deionized H₂O 5 μL and 4 μL of the extracted DNA. The reaction system is then placed into a 96-well plate. The 96-well plate is placed into a real-time fluorescent quantitative PCR device for reaction, including predenaturation at 94° C. for 10 min, denaturation at 94° C. for 30 s, annealing at a suitable annealing temperature for 30 s, extension at 72° C. for 45 s, for 45 cycles; and lastly, extension at 72° C. for 10 min. Once this is complete, the number of copies of genes of the various genera of bacteria in the samples can be calculated. In combination with the amplification curve of the standard sample, the relative abundance of the various strains in samples can be obtained.

### Analyzing Method

Once the sequencing is complete, the data can be analyzed. QIIME can be used for splitting the barcode of the raw data. Trimmomatic can be used for the quality control. FLASH can be used for the merger of the sequence data of the two ends of the sequence. Fastx Toolkit can be used to carry out a second quality control. Lastly, QIIME can be used again to remove the chimera in order to obtain clean reads. Parallel-Meta 3 can be used to carry out downstream OUT picking and then counting and analysis. The main parameters in the process include: Trimmomatic:SLIDINGWINDOW:30:25MINLEN:25; FLASH:-M 200-m 5-x 0.1; Fastx Toolkit:-Q 33-q 25-p 80; and Parallel-Meta 3:-L 123456.

### Microbial Index of Skin Health Method

The MiSH model was developed as discussed above, through the use of a random forest model and data from healthy skin and skin afflicted with atopic dermatitis (both lesion and non-lesion). From this, 25 bacteria were identified as having the ability to help determine if a skin sample was afflicted with a skin condition, like atopic dermatitis, and whether a composition can be used to treat the target condition effectively. These bacteria include: Fusobacterium, Capnocytophaga, Haemophilus, Comamonas, Kocuria, Carica, Streptococcus, Brachybacterium, Acinetobacter, Moraxella, Neisseria, Prevotella, Bergeyella, Rhizobium, Porphyromonas, Paenibacillus, Rothia, Wautersiella, Bacillus, Chryseobacterium, Deinococcus, Citrullus, Streptophyta Group, Paracoccus and Staphylococcus. This model is available online at www.single-cell.cn/skin. A graphical report of the MiSH will appear when a set of 16S rRNA sequencing data is input by following the model usage instruction, which is available at the website.

### Wash Protocol Method

The Wash Protocol Method includes two phases: a prewash phase and a treatment phase. This prewash phase is to normalize the skin condition by using the same washing product for all the participants for a certain period of time. The pre-wash product can be selected from a bar soap, a liquid body wash, a wipe, a powder cleanser, or water only. Preferably, the prewash product is a mild cleanser that would not cause any irritation or damage to the skin. An example is Olay Sensitive Skin Unscented Beauty Bar. The participants are, optionally, restrained from using any leave-on products (moisturizing lotion, sunscreens or beauty cosmetics) during the pre-wash phase to minimize potential interference from the leave-on products. The pre-wash period can last from one day to about 30 days, as is determined by the tester. Preferably, the prewash phase can last from 3 days to 21 days. Even more preferably, the prewash phase can last from about 5 days to about 14 days. Most preferably, the prewash phase can last about 7 days to about 10 days. At the end of the prewash phase, the baseline Microbial Skin Health Index is taken. The skin can be evaluated based on visual assessment and biophysical methods.

During the treatment phase, the participant will use a pre-assigned cleansing product. Optionally, the treatment phase may include a cleansing product and a leave-on treatment product. The treatment phase can last from about 1 day to about 90 days. Preferably, the treatment phase can last from 7 days to about 60 days. Preferably, the treatment can last from about 14 days to about 40 days. Most preferably, the treatment phase can last about 28 days. At the end of the treatment phase, the Microbial Index of Skin Health is taken. Optionally, the skin is evaluated based on visual assessment and bio-physical methods. The Microbial Index of Skin Health at the end of the treatment phase is compared to the Microbial Index of Skin Health at the baseline. An improvement of 5 points or higher is indicative of the skin health benefit from the treatment product.

### Combinations

A. A method of evaluating a skin cleansing composition for an ability to treat a skin condition, comprising: a) identifying a target skin condition or lack of target skin condition on a skin sample; b) performing a pre-wash protocol with a mild cleansing product via the Wash Protocol Method; c) taking a baseline measurement of a Microbial index of Skin Health via a Microbial index of Skin Health Method; d) performing a treatment wash protocol with the cleansing composition via the Wash Protocol Method; and e) taking a second measurement of the Microbial Skin Health Measurement after the Wash Protocol; wherein an increase from the baseline Microbial Skin Health Index of 5 or more signifies the cleansing product is efficacious for treatment of the identified skin condition.
B. The method of paragraph A, wherein the skin condition comprises dry skin, itchy skin, atopic dermatitis, sensitive skin, or a combination thereof.
C. The method of any of paragraphs A-B, wherein the skin condition is identified by swabbing a target area of skin to collect skin cells and testing the skin cells for a skin condition.
D. A method of evaluating a skin cleansing composition for an ability to treat a skin condition, comprising: a) prewashing a skin site from which a sample will be collected; b) collecting a prewash skin sample from the prewashed site as a baseline; c) sequencing the prewash skin sample for its bacterial population to produce prewash bacterial population data; d) reviewing the prewash bacterial population data for the abundance of the following bacteria: Fusobacterium, Capnocytophaga, Haemophilus, Comamonas, Kocuria, Carica, Streptococcus, Brachybacterium, Acinetobacter, Moraxella, Neisseria, Prevotella, Bergeyella, Rhizobium, Porphyromonas, Paenibacillus, Rothia, Wautersiella, Bacillus, Chryseobacterium, Deinococcus, Citrullus, Streptophyta Group, Paracoccus and Staphylococcus; e) assigning the relative abundances of the prewash bacteria population a number from 1-100; f) washing the skin site with the skin cleansing composition; g) collecting a post wash skin sample; h) sequencing the post wash skin sample for its bacterial population to produce post wash bacterial population data; i) reviewing the post wash bacterial population data for the abundance of the following bacteria: Fusobacterium, Capnocytophaga, Haemophilus, Comamonas, Kocuria, Carica, Streptococcus, Brachybacterium, Acinetobacter, Moraxella, Neisseria, Prevotella, Bergeyella, Rhizobium, Porphyromonas, Paenibacillus, Rothia, Wautersiella, Bacillus, Chryseobacterium, Deinococcus, Citrullus, Streptophyta Group, Paracoccus and Staphylococcus; j) assigning the relative abundance of the post wash bacteria population a number from 1-100; k) comparing the prewash bacteria population number to the post wash bacteria population number, wherein an increase in the post wash bacteria population number over the prewash bacteria population number of 5 or more signifies the cleansing composition is efficacious for treatment of the skin condition.
E. The method of paragraph D, wherein the prewashing step comprises washing the skin site with a specified cleanser for at least 3 days before the prewash skin sample is collected.
F. The method of any of paragraphs D-E, wherein sequencing the prewash skin sample is done via a 16S rRNA sequencing method.
G. The method of paragraph F, wherein the 16S rRNA sequencing method includes V1-V3 regions.
H. The method of any of paragraphs D-G, wherein the assigning of the relative abundances of the prewash bacteria population is done via www.single-cell.cn/skin.
I. The method of any of paragraphs D-H, wherein the assigning of the relative abundances of the prewash bacteria population is done via Parallel-Meta 3.
J. The method of any of paragraphs D-I, wherein the post washing step comprises washing the skin site with the skin cleansing composition for at least 3 days before the post wash skin sample is collected.
K. The method of any of paragraphs D-J, wherein sequencing the post wash skin sample is done via a 16S rRNA sequencing method.
L. The method of any of paragraphs D-K, wherein the 16S rRNA sequencing method includes V1-V3 regions.
M. The method of any of paragraphs D-L, wherein the assigning of the relative abundances of the post wash bacteria population is done via www.single-cell.cn/skin.
N. The method of any of paragraphs D-L, wherein the assigning of the relative abundances of the post wash bacteria population is done via Parallel-Meta 3.
O. A method of evaluating a leave-on skin composition for an ability to treat a skin condition, comprising: i) identifying a target skin condition or lack of target skin condition on a skin sample; ii) performing a pre-wash protocol with a mild cleansing product via the Wash Protocol Method; iii) taking a baseline measurement of a Microbial index of Skin Health via a Microbial index of Skin Health Method; iv) performing a treatment protocol with the leave-on skin composition wherein the leave-on skin composition is applied to the skin at least once a day; and v) taking a second measurement of the Microbial Skin Health Measurement after treatment with the leave-on skin composition; wherein an increase from the baseline Microbial Skin Health Index of 5 or more signifies the leave-on skin composition is efficacious for treatment of the identified skin condition.
P. The method of paragraph O, wherein the skin condition comprises dry skin, itchy skin, atopic dermatitis, sensitive skin, or a combination thereof.
Q. The method of any of paragraphs O-P, wherein the skin condition is identified by swabbing a target area of skin to collect skin cells and testing the skin cells for a skin condition.
R. A method of evaluating a leave-on skin composition for an ability to treat a skin condition, comprising: a) prewashing a skin site from which a sample will be collected; b) collecting a prewash skin sample from the prewashed site as a baseline; c) sequencing the prewash skin sample for its bacterial population to produce prewash bacterial population data; d) reviewing the prewash bacterial population data for the abundance of the following bacteria: Fusobacterium, Capnocytophaga, Haemophilus, Comamonas, Kocuria, Carica, Streptococcus, Brachybacterium, Acinetobacter, Moraxella, Neisseria, Prevotella, Bergeyella, Rhizobium, Porphyromonas, Paenibacillus, Rothia, Wautersiella, Bacillus, Chryseobacterium, Deinococcus, Citrullus, Streptophyta Group, Paracoccus and Staphylococcus; e) assigning the relative abundances of the prewash bacteria population a number from 1-100; f) applying the leave-on skin composition to the skin site; g) collecting a post application skin sample; h) sequencing the post application skin sample for its bacterial population to produce post wash bacterial population data; i) reviewing the post application bacterial population data for the abundance of the following bacteria: Fusobacterium, Capnocytophaga, Haemophilus, Comamonas, Kocuria, Carica, Streptococcus, Brachybacterium, Acinetobacter, Moraxella, Neisseria, Prevotella, Bergeyella, Rhizobium, Porphyromonas, Paenibacillus, Rothia, Wautersiella, Bacillus, Chryseobacterium, Deinococcus, Citrullus, Streptophyta Group, Paracoccus and Staphylococcus; j) assigning the relative abundance of the post application bacteria population a number from 1-100; k) comparing the prewash bacteria population number to the post application bacteria population number, wherein an increase in the post application bacteria population number over the prewash bacteria population number of 5 or more signifies the leave-on skin composition is efficacious for treatment of the skin condition.
S. The method of paragraph R, wherein the prewashing step comprises washing the skin site with a specified cleanser for at least 3 days before the prewash skin sample is collected.
T. The method of any of paragraphs R-S, wherein sequencing the prewash skin sample is done via a 16S rRNA sequencing method.
U. The method of paragraph T, wherein the 16S rRNA sequencing method includes V1-V3 regions.
V. The method of any of paragraphs R-U, wherein the assigning of the relative abundances of the prewash bacteria population is done via www.single-cell.cn/skin.
W. The method of any of paragraphs R-U, wherein the assigning of the relative abundances of the prewash bacteria population is done via Parallel-Meta 3.
X. The method of any of paragraphs R-W, wherein the post application step comprises applying the leave-on skin composition to the skin site at least once a day for at least 3 days before the post application skin sample is collected.
Y. The method of any of paragraphs R-X, wherein sequencing the post application skin sample is done via a 16S rRNA sequencing method.
Z. The method of paragraph Y, wherein the 16S rRNA sequencing method includes V1-V3 regions.
AA. The method of any of paragraphs R-Z, wherein the assigning of the relative abundances of the post wash bacteria population is done via www.single-cell.cn/skin.
BB. The method of any of paragraphs R-Z, wherein the assigning of the relative abundances of the post wash bacteria population is done via Parallel-Meta 3.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A method of evaluating a skin cleansing composition for an ability to treat a skin condition, comprising:
a. identifying a target skin condition or lack of target skin condition on a skin sample;
b. performing a pre-wash protocol with a mild cleansing product via the Wash Protocol Method;
c. taking a baseline measurement of a Microbial index of Skin Health via a Microbial index of Skin Health Method;
d. performing a treatment wash protocol with the cleansing composition via the Wash Protocol Method; and
e. taking a second measurement of the Microbial Skin Health Measurement after the Wash Protocol;
wherein an increase from the baseline Microbial Skin Health Index of 5 or more signifies the cleansing product is efficacious for treatment of the identified skin condition.

2. The method of claim 1, wherein the skin condition comprises dry skin, itchy skin, atopic dermatitis, sensitive skin, or a combination thereof.

3. The method of claim 1, wherein the skin condition is identified by swabbing a target area of skin to collect skin cells and testing the skin cells for a skin condition.

4. A method of evaluating a skin cleansing composition for an ability to treat a skin condition, comprising:
a. prewashing a skin site from which a sample will be collected;
b. collecting a prewash skin sample from the prewashed site as a baseline;
c. sequencing the prewash skin sample for its bacterial population to produce prewash bacterial population data;
d. reviewing the prewash bacterial population data for the abundance of the following bacteria: Fusobacterium, Capnocytophaga, Haemophilus, Comamonas, Kocuria, Carica, Streptococcus, Brachybacterium, Acinetobacter, Moraxella, Neisseria, Prevotella, Bergeyella, Rhizobium, Porphyromonas, Paenibacillus, Rothia, Wautersiella, Bacillus, Chryseobacterium, Deinococcus, Citrullus, Streptophyta Group, Paracoccus and Staphylococcus;
e. assigning the relative abundances of the prewash bacteria population a number from 1-100;
f. washing the skin site with the skin cleansing composition;
g. collecting a post wash skin sample;
h. sequencing the post wash skin sample for its bacterial population to produce post wash bacterial population data;
i. reviewing the post wash bacterial population data for the abundance of the following bacteria: Fusobacterium, Capnocytophaga, Haemophilus, Comamonas, Kocuria, Carica, Streptococcus, Brachybacterium, Acinetobacter, Moraxella, Neisseria, Prevotella, Bergeyella, Rhizobium, Porphyromonas, Paenibacillus, Rothia, Wautersiella, Bacillus, Chryseobacterium, Deinococcus, Citrullus, Streptophyta Group, Paracoccus and Staphylococcus;
j. assigning the relative abundance of the post wash bacteria population a number from 1-100;
k. comparing the prewash bacteria population number to the post wash bacteria population number, wherein an increase in the post wash bacteria population number over the prewash bacteria population number of 5 or more signifies the cleansing composition is efficacious for treatment of the skin condition.

5. The method of claim 4, wherein the prewashing step comprises washing the skin site with a specified cleanser for at least 3 days before the prewash skin sample is collected.

6. The method of claim 5, wherein sequencing the prewash skin sample is done via a 16S rRNA sequencing method.

7. The method of claim 6, wherein the 16S rRNA sequencing method includes V1-V3 regions.

8. The method of claim 7, wherein the assigning of the relative abundances of the prewash bacteria population is done via www.single-cell.cn/skin.

9. The method of claim 7, wherein the assigning of the relative abundances of the prewash bacteria population is done via Parallel-Meta 3.

10. The method of claim 4, wherein the post washing step comprises washing the skin site with the skin cleansing composition for at least 3 days before the post wash skin sample is collected.

11. The method of claim 10, wherein sequencing the post wash skin sample is done via a 16S rRNA sequencing method.

12. The method of claim 11, wherein the 16S rRNA sequencing method includes V1-V3 regions.

13. The method of claim 10, wherein the assigning of the relative abundances of the post wash bacteria population is done via www.single-cell.cn/skin.

14. The method of claim 10, wherein the assigning of the relative abundances of the post wash bacteria population is done via Parallel-Meta 3.

15. A method of evaluating a leave-on skin composition for an ability to treat a skin condition, comprising:
a. identifying a target skin condition or lack of target skin condition on a skin sample;
b. performing a pre-wash protocol with a mild cleansing product via the Wash Protocol Method;
c. taking a baseline measurement of a Microbial index of Skin Health via a Microbial index of Skin Health Method;
d. performing a treatment protocol with the leave-on skin composition wherein the leave-on skin composition is applied to the skin at least once a day; and
e. taking a second measurement of the Microbial Skin Health Measurement after treatment with the leave-on skin composition;
wherein an increase from the baseline Microbial Skin Health Index of 5 or more signifies the leave-on skin composition is efficacious for treatment of the identified skin condition.

16. The method of claim 1, wherein the skin condition comprises dry skin, itchy skin, atopic dermatitis, sensitive skin, or a combination thereof.

17. The method of claim 1, wherein the skin condition is identified by swabbing a target area of skin to collect skin cells and testing the skin cells for a skin condition.
